(19) United States
(12) Patent Application Publication
(10) Pub. No.: US 2003/0221211 A1
(43) Pub. Date: Nov. 27, 2003

(54) Methods of suppressing gene expression

(75) Inventors: William H. Rottmann, Summerville, SC (US); Richard L.S. Forster, Auckland (NZ)

Correspondence Address: FOLEY AND LARDNER, SUITE 500, 3000 K STREET NW, WASHINGTON, DC, 20007, US

(73) Assignee: ArborGen, LLC

(21) Appl. No.: 10/354,152

(22) Filed: Jan. 30, 2003

Related U.S. Application Data
(60) Provisional application No. 60/352,247, filed on Jan. 30, 2002.

(51) Int. Cl. A01H 1/00; C12N 15/82; C12N 5/04

(52) U.S. Cl. 800/278; 435/468; 435/419; 435/320.1

(57) ABSTRACT

DNA constructs useful for suppressing the expression of targeted genes are provided. The constructs encode an RNA molecule with a stem-loop structure that is unrelated to the target gene and that is positioned distally to a sequence specific for the gene of interest. Methods of suppressing the expression of selected genes and transformed hosts also are described. In one embodiment, the DNA constructs and methods are used to selectively suppress gene expression in plants and plant cells.

This application claims the priority benefit of U.S. Provisional Application Serial No. 60/352,247, filed on Jan. 30, 2002, which is hereby incorporated by reference.

### FIELD OF THE INVENTION

The present invention relates to novel DNA constructs used to suppress the expression of targeted genes. The invention also relates to methods of suppressing gene expression using the constructs described herein. In one embodiment, the DNA constructs and methods are used to suppress gene expression in plants and plant cells.

### BACKGROUND OF THE INVENTION

Reduction of the activity of specific genes (also known as gene silencing or gene suppression) is desirable for several aspects of genetic engineering in plants, including testing for the functions of uncharacterized genes, inactivating reproductive genes to produce sterile plants, and inactivating genes for biosynthetic enzymes to reduce the lignin content of wood.

Placing a selected coding region under the control of a strong promoter in transgenic plants has led to turning off the gene(s) that have a strong sequence similarity to the introduced coding region. This field of research has been reviewed thoroughly in recent years (e.g., Mol et al., 1994. in Homologous Recombination and Gene Silencing in Plants. J. Paszkowski, ed. pp. 309-334; Fagard and Vaucheret, 2000. Annu. Rev. Plant Physiol. Plant Mol. Biol. 51: 167-194.). Silencing can be at the point of transcription—possibly caused by methylation of the target DNA (Wassenegger. 2000. Plant Mol. Biol. 43: 203-220), or post-transcriptional—through rapid degradation of target RNA (Meins. 2000. Plant Mol. Biol. 43: 261-273). Although the details of these mechanisms are not fully understood, recent evidence strongly suggests that double-stranded RNA is the primary initiator of the targeted RNA degradation process (e.g., Lipardi et al., 2001. Cell 107: 297-308; Sijen et al., 2001. Cell 107: 465-476, Mette et al., 2000. Embo. J. 19:5194-5201.).

Gene silencing or gene suppression has been produced by transgenes with the coding region in either the sense orientation (Napoli et al., 1990. Plant Cell 2: 279-289; Jorgensen et al., 1991. U.S. Pat. No. 5,034,323.) or the antisense orientation (Ecker and Davis, 1987. PNAS 83: 5372-5376; Shewmaker et al., 1991. U.S. Pat. No. 5,197,065.) relative to the direction of transcription. Suppression by simple sense or antisense constructs is often inefficient. For example, sense suppression usually occurs in about 5% to 20% of transformation events (Mol et al., 1994). Numerous papers describe increased frequency of gene silencing when complementary (sense and antisense) RNAs are present together (for example, Waterhouse, et al., 1998. PNAS 95: 13959-13964; Chang and Meyerowitz, 2000. PNAS 97: 4985-4990; Levin et al., 2000. Plant Mol. Biol. 44: 759-775.).

Gene silencing that is induced by double-stranded RNA is commonly called RNA interference or RNAi. A generally recognized hallmark of RNAi is the presence of short RNA segments (21-25 nucleotides long) that match sequences in the silenced gene (Hamilton and Baulcombe, 1999. Science 286:950-952.) These small interfering RNAs (siRNAs) are believed to participate in identifying complementary transcripts for degradation.

Most suppression methods based on double-stranded RNA (dsRNA) are ill suited for functional screening many different genes because the effort of putting the target gene sequence into both sense and antisense is too great. One method that overcomes this hurdle is the use of virus-based constructs to produce double-stranded RNA (Baulcombe et al., 1999. PCT application WO 99/15682). However, suppression induced by viruses does not always appear to be effective in meristems and shoot tips (Dalmay et al., 2000. Plant Cell 12: 369-379.). Also, there are regulatory impediments to use of virus-based suppression in plants grown outdoors, for example large species such as trees.

An improvement has involved the use of a bacterial recombinase to insert a selected sequence into a plasmid-based expression vector in an inverted repeat conformation without performing multiple cloning steps (Wesley et al., 2001. Plant J. 27: 581-590.). However, this method still requires that each gene segment be amplified and inserted separately, so it can not be used to create a random library of suppression constructs directly from a mixture of cDNAs.

It has also been proposed that linking a double-stranded RNA region to a target gene RNA sequence would lead to silencing of the target gene (Grierson et al., 1998. WO 98/53083; Sheen, 2000. WO 00/49035). However, recent evidence shows that refinement is needed. For example, cloning a target sequence between two units of an unrelated inverted repeat does not lead to silencing of the target gene (Levin et al., 2000.) Additionally, experiments in our laboratories have found that the simple presence of a stem-loop structure derived from an unrelated sequence does not lead to silencing of a linked target gene. It seems likely that the successes reported in the cited applications are due only to the fact that the stem-loops were of the same or similar sequence as the targets.

A second inducer of gene silencing that has been repeatedly proposed is a threshold level of aberrant transcripts, for example those that have been improperly processed (Fagard and Vaucheret, 2000). Chua and van der Krol (1997, U.S. Pat. No. 5,686,649) described a petunia chalcone synthase construct in which a 3′-processing signal downstream of the poly(A) addition site was deleted. However, the silencing of gene activity by such constructs was not very efficient.

Accordingly, there is still a need for constructs that induce a gene suppression mechanism against a wide selection of target genes and that result in complete or near-complete silencing of expression at high efficiency.

### SUMMARY OF THE INVENTION

It is, therefore, one object of the present invention to provide DNA constructs useful for suppressing the expression of targeted genes.

It is also an object of the present invention to provide methods of suppressing the expression of targeted genes.

Another object of the invention is to provide hosts, in particular plants, transformed with DNA constructs useful for suppressing the expression of targeted genes.

In accomplishing these and other objects of the invention, there is provided, in accordance with one aspect of the invention, a DNA construct comprising a promoter operably linked to a first DNA segment that corresponds to at least a portion of a target gene, a second DNA segment that corresponds to at least a portion of a gene different from the target gene, a third DNA segment that has substantial sequence identity to the second DNA segment, a spacer, and a fourth DNA segment that is the reverse complement of at least a portion of the third DNA segment, wherein the first, second, third and fourth DNA segments are arranged in a 5′ to 3′ direction, respectively, in the DNA construct. In one embodiment, the first DNA segment comprises a sense sequence, while in another an anti-sense sequence. In another embodiment, the target gene is a promoter or transcription regulatory region of a gene of interest.

In still another embodiment, there is provided an expression system comprising A) a first DNA cassette comprising a first DNA segment that corresponds to at least a portion of a target gene and a second DNA segment that corresponds to at least a portion of a gene different from the target gene, wherein the first and second DNA segments are arranged in a 5′ to 3′ direction, respectively, in the first DNA cassette, B) a second DNA cassette comprising a third DNA segment that has substantial sequence identity to the second DNA segment of the first DNA cassette, a spacer and a fourth DNA segment that is the reverse complement of at least a portion of the third DNA segment, wherein the third and fourth DNA segments are arranged in a 5′ to 3′ direction, respectively, in the first DNA cassette.

In another embodiment, the invention provides a DNA construct comprising a promoter operably linked to a first DNA segment that corresponds to at least a portion of a promoter of a target gene, a spacer, and a second DNA segment that is the reverse complement of at least a portion of the first DNA segment, wherein the first and second DNA segments are arranged in a 5′ to 3′ direction, respectively, in the DNA construct.

The invention also provides a vector useful for suppressing a targeted gene utilizing a DNA segment, unrelated to the targeted gene, that contains a defective intron. In other embodiments, the invention provides vectors useful for suppressing a targeted gene utilizing double-stranded RNA technology where the nucleotide sequence of the double-stranded RNA is not related to the nucleotide sequence of the target gene. This unrelated double-stranded RNA segment can include any portion of the coding sequence, an intron, the promoter or any untranslated region of the unrelated gene.

The invention further provides methods of suppressing the expression of a target gene in a cell comprising introducing into the cell the DNA constructs disclosed herein. Transformed cells and organisms also are provided herein. In a preferred embodiment, the invention relates to transgenic plants comprising the inventive constructs.

Other objects, features and advantages of the present invention will become apparent from the following detailed description. The detailed description and specific examples, while indicating preferred embodiments, are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIGS. 1A-C depicts general maps for suppression constructs comprising a multiple cloning site and a defective intron. The defective intron is represented by a white rectangle with an X at one end. A. Cassette with a multiple cloning site for insertion of target gene segments. B. Cassette with a target gene segment inserted upstream of the defective intron. C. Cassette with two target gene segments.

FIGS. 2A-D depicts several general maps for suppression constructs comprising an inverted repeat of an unrelated gene sequence. A. Cassette comprising a multiple cloning site and an inverted repeat of an unrelated gene segment. B. Cassette comprising a target gene segment in front of an inverted repeat of an unrelated gene segment. C. Cassette in which the inverted repeat is extended into the 3′-UTR of the terminator by the addition of an antisense copy of the 3′-UTR in front of the original inverted repeat. D. Cassette in which the inverted repeat is extended into the poly(A) tail by the addition of an oligo(dT) segment in front of the antisense 3′-UTR.

FIG. 3 depicts a construct comprising a target DNA segement joined to a DNA segment from an unrelated gene repeated three times, twice as a direct repeat and once in the opposite orientation.

FIGS. 4A-C depicts maps for cassettes that are combined to produce suppression. A. Cassette comprising a target gene sequence and segment from unrelated gene, acting to transfer suppression to the target DNA segment. B. Cassette comprising an inverted repeat of a DNA segment from an unrelated gene, designed to induce the suppression mechanism. C. Plasmid with cassette comprising an inverted repeat from LSAG plus cassette comprising GUS target gene sequence and segment from LSAG.

FIG. 5 depicts a general map for a suppression cassette comprising an inverted repeat of a DNA segment from the promoter of a target gene.

FIG. 6 depicts the method of constructing pWVR108, a high copy plasmid with a suppression cassette comprising a GUS target gene segment and a defective intron from a sweetgum gene. Gene components are labeled. The white segments indicate introns; the defective intron is indicated with an X.

FIG. 7A depicts the map of plasmid pWVR110, a binary plasmid comprising a construct for suppressing GUS that is based on a defective intron. The white segments indicate introns; the defective intron is indicated with an X. FIG. 7B depicts the map of plasmid pWVr8.

FIGS. 8A-B depicts a method for constructing pWVR128, a high copy plasmid comprising a cassette for suppressing GUS that is based on an inverted repeat of an unrelated sweetgum sequence. A. Cloning of component segments. B. Insertion of inverted repeat into GUS cassette. Gene components are labeled. The white segments indicate introns.

FIG. 9 depicts the method of constructing pWVR77, a high copy plasmid with a GUS suppression cassette comprising a DNA segment from an unrelated sweetgum gene repeated three times, twice as a direct repeat and once in the opposite orientation. The white segments indicate introns.

FIGS. 10A-B depicts binary vectors. A. pWVR5, a compact binary vector with selectable marker. B. pWVR79, a binary vector suitable for suppression of random target genes by inserting segments of the target genes in front of a triply repeated DNA segment from an unrelated gene. The white segments indicate introns.

FIGS. 11A-B depicts the construction of two cassettes that, when combined in one plant, result in silencing of the target gene. A. Construction of a cassette that produces double-stranded RNA of a segment from an unrelated gene. B. Construction of a cassette that links the same segment of the unrelated gene 3′ to the target sequence. The white segments indicate introns.

FIG. 12 depicts the construction of a cassette comprising an inverted repeat of a segment from the Arabidopsis ACTIN2 promoter. The white segment indicates an intron in the 5′-UTR of the UBQ3 gene.

FIG. 13 shows the GUS staining of samples from transformed tobacco containing a control plasmid, a construct with GUS linked to a simple inverted repeat of an unrelated sequence, and a construct with GUS linked to an unrelated sequence repeated three times, twice as a direct repeat and once in the opposite orientation.

### DETAILED DESCRIPTION

The present invention provides DNA constructs useful for suppressing the expression of targeted genes. The DNA constructs can be employed in any type of eukaryotic cell or organism, including plant and animal cells. Additionally, the constructs and methods described herein can be used in individual cells in culture, or in vivo in organisms, or in organs or other portions of organisms.

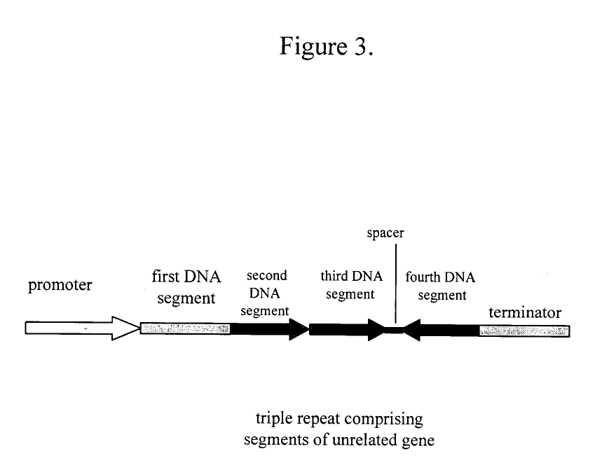

The inventive constructs selectively suppress target genes by encoding double-stranded RNA (dsRNA) and initiating RNA interference (RNAi). However, unlike previous methods which employed constructs that generated dsRNA comprising the actual sequence of the gene selected for silencing, the constructs of the present invention generate RNA molecules with a stem-loop structure that is unrelated to the target gene. The stem-loop structure is located on the distal end of the generated RNA and follows a single stranded sequence that is specific for the target gene. Separating the target-specific sequence on the proximal end and the stem-loop structure on the distal end is a nucleotide sequence with substantial sequence identity to the first “stem” of the stem-loop structure. The general structure of this embodiment is shown in FIG. 3.

In another aspect of the invention, DNA constructs are provided which deliver, either directly or indirectly, dsRNA corresponding to the promoter of a target gene. Upon binding to the promoter, the dsRNA induces gene suppression of the target gene.

Definitions

The phrases “target gene” and “gene of interest” are used interchangeably herein. Target gene, as understood in the current context, is used to mean the gene that is pinpointed for suppression. The targeted gene may or may not contain regulatory elements such as, for example, a transcription factor binding site or enhancer. Genes that can be chosen for suppression include those that code for structural proteins, such as cell wall proteins, or for regulatory proteins such as transcription factors and receptors, as well as other functional genes. Furthermore, the term is meant to include not only the coding region of a polypeptide but also introns present in the DNA, regulatory elements, the promoter and the transcription terminator. Thus, “at least a portion of the target gene” is meant to include at least a portion of the transcribed sequence and/or at least a portion of the promoter and/or at least a portion of the terminator of the gene of interest.

The DNA constructs of the present invention comprise, at their most basic level, a promoter, one or more DNA segments and a transcription terminator. As used herein, “DNA segment” is meant to refer to a deoxyribonucleic acid molecule comprised of at least several contiguous bases. The DNA segment that corresponds to the target gene may be 30 base pairs (bp) or greater in length, preferably at least 50 bp and less than 2000 bp, and more preferably at least 100 bp and less than 750 bp. The DNA segment can be single- or double-stranded. A DNA segment, within the context of the present invention, can include a gene or cDNA or a portion thereof, or it can include a promoter or a regulatory element or a portion thereof.

The term “spacer” refers to a series of contiguous bases that do not code for any portion of a gene or promoter or regulatory element, such as an entirely synthetic sequence or a region between genes. A “spacer DNA segment” codes for a “spacer RNA segment,” and merely serves to separate other DNA or RNA segments. The length of a spacer may vary over a wide range, from 10 base pairs (bp) to 2000 bp or more. When very long complementary segments of DNA are separated by a short spacer, the construct may be unstable. Therefore, the spacer preferably should be between ¼ to 2 times the length of the segments it is separating. For example, if complementary DNA segments of 160 bp are present, the spacer segment between them would preferably be between 40 to 320 bp. The spacer may encode an intron that is spliced out of the transcript so that the resulting spacer RNA is much shorter than the complementary DNA segments of the transcript. Thus, a spacer DNA segment codes for a “spacer RNA segment,” which merely serves to separate other RNA segments.

The term “RNA segment” refers to a ribonucleic acid molecule comprised of at least several contiguous bases. The RNA segment may be a transcript, i.e. an mRNA molecule that codes for an entire polypeptide, or it may be a portion thereof. Furthermore, the RNA segment need not code for a polypeptide or any portion thereof, as long as the segment meets the qualities of RNA segment defined herein. For example, an RNA segment may comprise an intron, a 5′-UTR, or a 3′-UTR, which do not encode peptides. An RNA segment also is produced when a DNA segment comprising a promoter, a regulatory element, or a non-gene sequence is transcribed.

Two DNA segments that have similar or identical sequences on opposite DNA strands are referred to as “inverted repeats.” Transcription through a region with inverted DNA repeats produces RNA segments that are “complementary” to each other. Complementary RNA or DNA segments are segments that will specifically bind to each other. A transcript that comprises two complementary segments of RNA can form a single RNA molecule with double-stranded regions. Such double-stranded regions are sometimes called “stem-loops” or “hairpins.”

Preferably, the sequence of two complementary segments should be at least 80% identical to each other. More preferably, the complementarity should be at least 85%, 90%, 95%, 96%, 97%, 98%, 99% or even 100%. The DNA segments that are complementary to each other may be 30 base pairs (bp) or greater in length, preferably at least 50 bp and less than 2000 bp, and more preferably at least 100 bp and less than 750 bp.

By 95% complementarity, for example, it is meant that nucleotides of the complementary RNA or DNA segments will bind to each other in an exact base-to-base manner, except that one RNA or DNA segment may contain up to 5 point mutations per 100 bases of the other complementary strand of the RNA or DNA segment. The point mutations may be in the form of a deleted base or a substituted base. Furthermore, these mutations of the reference sequence may occur at the 5′ or 3′ terminal positions of one of the complementary nucleotide sequences or anywhere between the terminal positions, interspersed either individually among nucleotides in the reference sequence or in one or more contiguous groups within the reference sequence.

As a practical matter, percent complementary can be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) which are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps. Alternatively, percent complementarity can be assessed using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

By “transcription terminator” is meant a segment of DNA that encodes the 3′-end of an RNA transcript that causes RNA polymerase to halt or retard transcription. Because most eukaryotic mRNAs have poly(A) segments added to their 3′-ends, most transcription terminators specify a base or bases to which adenosyl residues are added. Thus, a transcription terminator can comprise DNA encoding at least a portion of the 3′-UTR of an mRNA immediately adjacent to and including the nucleotide(s) to which a poly(A) tail is added. A transcription terminator additionally can comprise at least a portion of the DNA sequence immediately after the site(s) of polyadenylation to provide a more complete DNA context for the transcription stop site. Transcription terminators also include segments that halt transcription other than terminators for polyadenylation such as transcription terminators for histone genes or ribosomal RNA genes.

DNA constructs, as used herein, also encompass vectors. The term “vector” refers to a DNA molecule capable of autonomous replication in a host cell. As known to those skilled in the art, a vector includes, but is not limited to, a plasmid, cosmid, phagemid, viral vectors, phage vectors, yeast vectors, mammalian vectors and the like. Typically, vectors will include a gene coding for a drug resistance marker, a thymidine kinase gene or a gene that complements an auxotroph. Various antibiotic resistance genes have been incorporated into vectors for the purpose of aiding selection of host cell clones containing such vectors. For example, antibiotic resistance genes incorporated into vectors intended for introduction into bacterial host cells include, but are not limited to, a gene that confers resistance to an antibiotic selected from the group consisting of ampicillin, kanamycin, tetracycline, neomycin, G418, blastocidin S and chloramphenicol. Genes for complementing an auxotroph are genes encoding enzymes or proteins which facilitate usage of nutritional or functional components by the host such as a purine, pyrimidine, amino acid (e.g., lysine, tryptophan, histidine, leucine, cysteine), or sphingolipid.

Additionally, vectors will include an origin of replication (replicons) for a particular host cell. Various prokaryotic replicons are known to those skilled in the art, and function to direct autonomous replication and maintenance of a recombinant molecule in a prokaryotic host cell.

The term “operably linked” refers to the chemical fusion, ligation, or synthesis of DNA such that a promoter-DNA sequence combination is formed in a proper orientation for the DNA sequence to be transcribed into an RNA segment. Transcription from the promoter-DNA sequence may be regulated by the promoter, possibly in combination with other regulatory elements. Alternatively, transcription from the promoter-DNA segment may not be regulated by the promoter. In the construction of the promoter-DNA sequence combination, it is generally preferred to position the promoter at a distance upstream from the initial codon of the DNA segment that is approximately the same as the distance between the promoter and the segment it controls in its natural setting. However, as known in the art, substantial variation in the distance can be accommodated without loss of promoter function.

The term “promoter” denotes a nucleotide sequence, natural or synthetic, capable of binding RNA polymerase to initiate transcription. Such promoters are known to those skilled in the art and may include bacterial, viral, fungal, plant, mammalian, or other eukaryotic promoters, the selection of which depends on the host cell or organism being transformed. It is expected that silencing of the target gene will be most effective when the suppressing construct is transcribed in the same tissue as the target gene. Although there is evidence that the silencing signal can be translocated to distant parts of a plant (e.g., Palauqui and Vaucheret, 1998. PNAS 95: 9675-9680.), some cells may not be able to receive such a signal. For example, GFP expression at the very tip of the growing shoot was not silenced by a viral suppression construct (Dalmay et al., 2000. Plant Cell 12: 369-379.). To achieve silencing of a gene expressed in many types of cells, a constitutive promoter of at least moderate strength is preferred. Examples of constitutive promoters that act in plants are viral promoters such as CaMV 35S or FiMV (Sanger et al., 1990. Plant Mol. Biol. 14: 433-443), bacterial promoters such as nopaline synthase (nos) or mannopine synthase (mas), or plant promoters such as those from the Arabidopsis ACTIN2 or UBIQUITIN10 genes (An et al., 1996. Plant J. 10: 107-121; Norris et al., 1993. Plant Mol. Biol. 21: 895-906). Target genes with limited expression patterns also can be silenced using a constitutive promoter to drive the suppression construct. However, it may be desirable to avoid expression of the suppression construct beyond what is necessary for the silenced phenotype. A promoter for the suppression construct could be used that has a pattern of expression similar to that of the target gene. For example, if silencing of a xylem-expressed target is planned, the promoter from the parsley 4CL gene (Hauffe et al., 1993. Plant J. 4: 235-253) could be used, or if a meristem-specific gene is targeted, the Arabidopsis PROLIFERA promoter (Springer et al., 1995. Science 268: 877-880) could be used. Preferably, the promoter should be derived from a different species than the species being transformed, to avoid interactions between identical promoter sequences. Various other promoters for expression in eukaryotic cells are known in the art, including, but not limited to, viral or viral-like basal promoters like the SV40 late promoter and the RSV promoter, and fungal or mammalian cellular promoters (see, e.g., Larsen et al., 1995, Nucleic Acids Res. 23:1223-1230; Donis et al., 1993, BioTechniques 15:786-787; Donda et al., 1993, Mol. Cell. Endocrinol. 90:R23-26; and Huper et al., 1992, In Vitro Cell Dev. Biol. 28A:730-734). Various replicons are known to those skilled in the art that function in eukaryotic cells to direct replication and maintenance of a recombinant molecule, of which it is part of, in a eukaryotic host cell.

The term “regulatory element” refers to nucleic acid sequences that affect the specificity or efficiency of DNA transcription or mRNA translation including, but not limited to, binding sites for transcription factors, enhancers, and transcription or translation initiation and termination signals. Enhancer sequences are DNA elements that appear to increase transcriptional efficiency in a manner relatively independent of their position and orientation with respect to a nearby DNA segment. Thus, depending on the DNA construct, an enhancer may be placed either upstream or downstream from a particular DNA segment to increase transcriptional efficiency. Such regulatory elements may be inserted into construct DNA sequences using recombinant DNA methods known in the art. Other regulatory elements include, but are not limited to, a 5′ untranslated region (5′UTR) on the RNA segment as well as a 3′UTR (i.e., comprising the poly(A) tail) on the RNA segment, which are necessary for stability and efficient translation of the RNA segment or transcript.

As used herein, “a gene different from the gene of interest,” or a “different gene” or an “unrelated gene” is a gene, or segment thereof, that does not share significant sequence identity with any portion of the gene targeted for suppression. This concept is contrasted with the phrase “not identical,” which simply means that two (or more) polynucleotides can not have identical sequences (i.e. 100% sequence identity), although they may share regions of sequence identity. Thus, as used in the current context, mRNA encoding the complement of a gene different from the gene of interest would not hybridize with mRNA encoding the gene of interest under stringent conditions. Whereas mRNA encoding the complement of a segment not identical to the gene of interest may be able to hybridize with MRNA encoding the gene of interest under stringent conditions. As envisioned in the present invention, the unrelated gene can be purely synthetic, or derived from an entirely different species.

A “defective intron,” as used herein, is an intron that is not properly spliced out the mRNA segment or transcript. Examples of defects in the intron include, but are not limited to, incomplete introns or a sequence mutation that does not allow proper excision of the intron.

As used herein, a “cassette” is a type of DNA construct comprising a promoter, a transcription terminator, and the DNA segments inserted between them. A cassette can be used to drive the expression of DNA or RNA segments in host cells or organisms in which the promoter is active.

The term “substantial sequence identity” describes the relatedness of two or more nucleotide sequences. Preferably, the sequences are at least 80% identical to each other, as calculated above. More preferably, the complementarity should be at least 85%, 90%, 95%, 96%, 97%, 98%, 99% or even 100%.

Discussion

The present invention provides DNA constructs useful for suppressing the expression of targeted genes. In one embodiment, there is provided a DNA construct comprising a promoter operably linked to a first DNA segment that corresponds to at least a portion of a target gene, a second DNA segment that corresponds to at least a portion of a gene different from the target gene, a third DNA segment that has substantial sequence identity to the second DNA segment, a spacer, and a fourth DNA segment that is the reverse complement of at least a portion of the third DNA segment, wherein the first, second, third and fourth DNA segments are arranged in a 5′ to 3′ direction, respectively, in the DNA construct (FIG. 3).

Thus, when transcribed, the inventive DNA constructs yield a RNA molecule comprising a first RNA segment corresponding to at least a portion of a target gene, a second RNA segment that corresponds to at least a portion of a gene different from the target gene, a third RNA segment that has substantial sequence identity to the second RNA segment, a spacer and a fourth RNA segment that is the reverse complement of at least a portion of the third RNA segment.

While the mechanism by which the invention operates is not fully understood, and the inventors do not wish to limit their invention to any particular theory, it is believed that the third and fourth RNA segments of the resulting RNA molecule form a stem-loop. The dsRNA of the stem loop likely is degraded into a first small interfering RNA (siRNA) of about 21-23 nucleotides in length. The first siRNA then associates with the second RNA segment due to their sequence homology. A dsRNA-dependent RNA polymerase then may recognize the complex and synthesize complementary RNA (cRNA), using the second and first nucleotide segments as a template. The resulting duplex, comprising the cRNA and the first and second nucleotide segments, may then be degraded to a second siRNA, which is specific to the target gene.

There is considerable leeway in the structural details of the triple repeat formed by the second, third, and fourth DNA segments. The repeated segments can have a number of differences relative to each other, as long as substantial sequence identity remains between the segments. In one embodiment, the repeated sequences exhibit 100% identity or 100% complementarity throughout their lengths, with differences confined to the ends of the segments.

The repeated segments of the unrelated gene can be in either sense or antisense (forward or reverse) orientation relative to their source gene. In FIG. 3, the segments of the unrelated gene (second, third and fourth DNA segments) are, in a 5′ to 3′ order, “forward, forward, and reverse”. An orientation order of “reverse, reverse, forward” is equally preferred. A key aspect of this embodiment is that the single-stranded RNA segment resulting from transcription of the triple repeat lies 3′ and adjacent to the RNA segment comprising the target sequence. In this case the RNA-dependent RNA polymerase that is part of the suppression machinery can extend newly synthesized complementary RNA (cRNA) from the single-stranded sequence to the target sequence using as primers the small RNAs resulting from degradation of the double-stranded RNA of the inverted repeat.

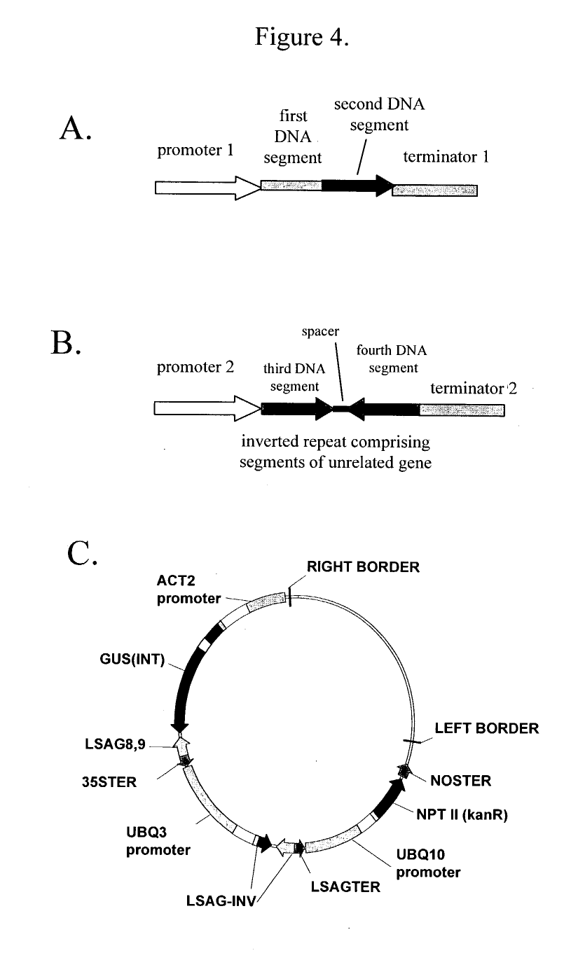

In another embodiment, there is provided an expression system comprising A) a first DNA cassette comprising a first DNA segment that corresponds to at least a portion of a target gene and a second DNA segment that corresponds to at least a portion of a gene different from the target gene, wherein the first and second DNA segments are arranged in a 5′ to 3′ direction, respectively, in the first DNA cassette, B) a second DNA cassette comprising a third DNA segment that has substantial sequence identity to the second DNA segment of the first DNA cassette, a spacer and a fourth DNA segment that is the reverse complement of at least a portion of the third DNA segment, wherein the third and fourth DNA segments are arranged in a 5′ to 3′ direction, respectively, in the first DNA cassette (FIG. 4).

In one embodiment of a combination of DNA constructs comprising a portion of the target gene, the first and second cassettes are on the same plasmid (FIG. 4C). In another embodiment, the first and second cassettes are on different plasmids, but both are inserted into the same organism or host cell. In still another embodiment, the first and second promoters are identical.

There is a significant amount of variation that is permissible in the structures of the cassettes in these combinations. The segment of the unrelated gene that is linked to the target gene segment may be either in the forward or reverse orientation relative to its source gene. Likewise, the segments of the unrelated gene in the inverted repeat may be either “forward, reverse” or “reverse, forward.” The repeated segments can have multiple differences relative to each other, as long as substantial sequence identity remains between the segments. Preferably, the sequence of two complementary segments should be at least 80% identical to each other. More preferably, the complementarity should be at least 85%, 90%, 95%, 96%, 97%, 98%, 99% or even 100%. In one embodiment, the repeated sequences exhibit 100% identity or 100% complementarity throughout their lengths, with differences confined to the ends of the segments.

The single-stranded RNA segment resulting from unrelated DNA segment should lie 3′ and adjacent to the RNA segment comprising the target sequence. In this case the RNA-dependent RNA polymerase that is part of the suppression machinery can extend newly synthesized cRNA from the single-stranded sequence to the target sequence in the first cassette using as primers the small RNAs resulting from degradation of the double-stranded RNA produced by the second cassette comprising the inverted repeat.

The invention also provides a combination of DNA constructs or a multi-cassette DNA construct comprising a first and second DNA construct (FIGS. 4A and 4B). The first DNA construct comprises a first promoter, a first DNA segment encoding a first RNA segment, a second DNA segment encoding a second RNA segment and a first transcription terminator. The second DNA construct comprises a transcribed inverted repeat of the second DNA segment, in the form: a second promoter, a third DNA segment encoding a third RNA segment, where the second and third DNA segments have substantial sequence identity with each other, a spacer DNA segment encoding an RNA spacer segment, a fourth DNA segment encoding a fourth RNA segment that is complementary to the third RNA segment and forms double-stranded RNA with the third RNA segment, and a second transcription terminator.

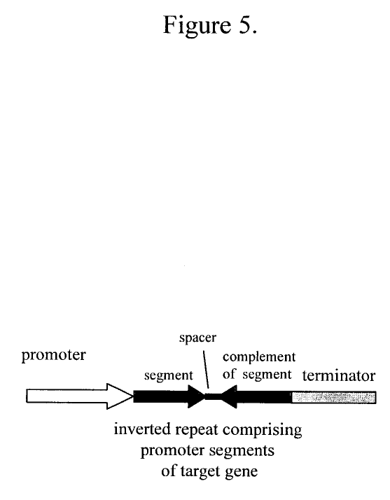

In still another embodiment, there is provided a DNA construct comprising a promoter operably linked to a first DNA segment that corresponds to at least a portion of a promoter of a target gene, a spacer and a second DNA segment that is the reverse complement of at least a portion of the first DNA segment, wherein the first and second DNA segments are arranged in a 5′ to 3′ direction, respectively, in the DNA construct (FIG. 5).

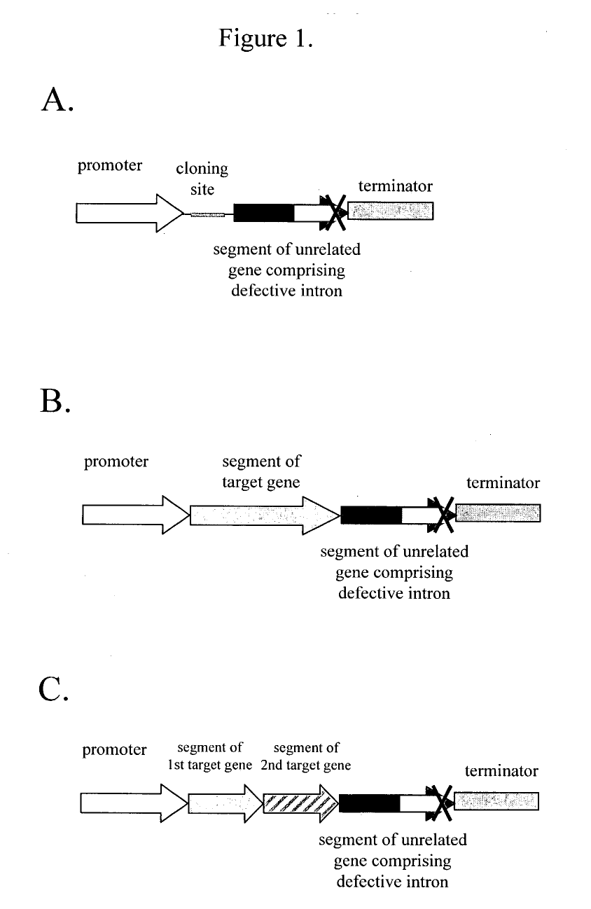

In another embodiment, the present invention provides a DNA construct comprising, in a 5′ to 3′ direction, a promoter, a multiple cloning site or a first DNA segment that codes for at least a portion of a gene of interest, a second DNA segment that codes for at least a portion of a gene different from the gene of interest, which comprises a defective intron, and a transcription terminator (FIGS. 1A and 1B). The first DNA segment may or may not contain an additional intron; and the second DNA segment may or may not contain an additional intron.

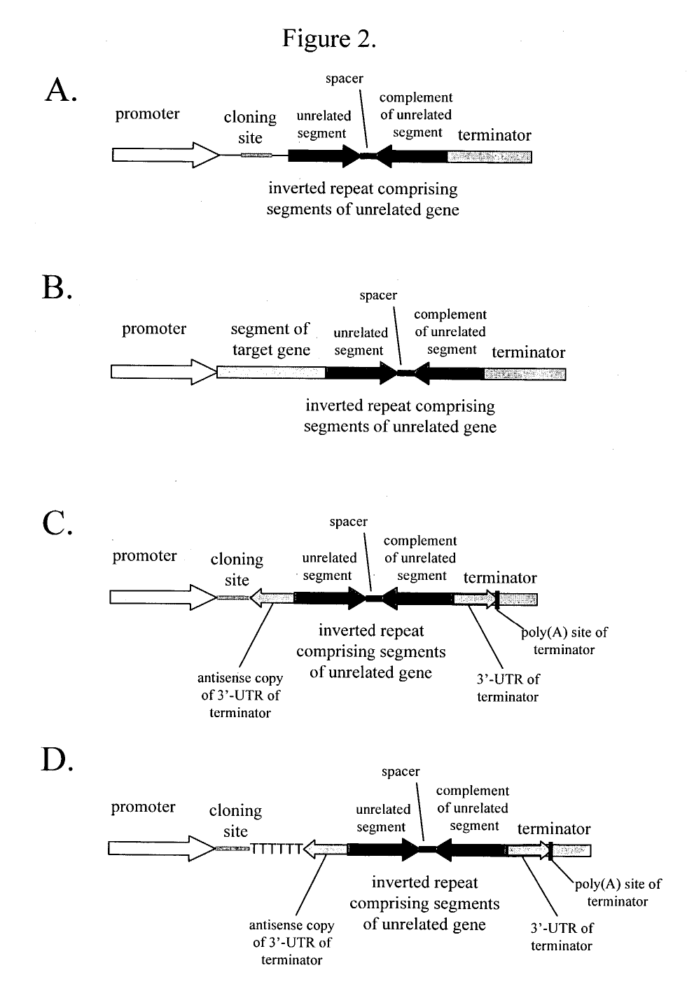

The invention also provides a DNA construct comprising, in a 5′ to 3′ direction, a promoter, a multiple cloning site or a DNA segment coding for at least a portion of the gene of interest, a first DNA segment encoding a first RNA segment, a spacer DNA segment encoding an RNA spacer segment, a second DNA segment encoding a second RNA segment, which is complementary to the first RNA segment and forms a double-stranded RNA molecule with the first RNA segment, and a transcription terminator (FIGS. 2A and 2B). The first and second DNA segments of the above-described construct are preferably DNA segments that are from a different species that the intended species of targeted gene suppression. For example, if gene suppression is targeted in a plant, the first and second DNA segments should preferably be from a different species, including but not limited to, other plant species, any animal species or bacterial species.

In one embodiment, the construct further comprises a DNA segment coding for a 3′ untranslated region (3′-UTR) complement that lies between the first DNA segment and the multiple cloning site (or DNA encoding at least a portion of the gene of interest), such that, when transcribed, it codes for an RNA segment that is complementary to the 3′-UTR region present in the transcription terminator used in the construct (FIG. 2C). The 3′ UTR and its complement contribute to the formation and lengthening of the double-stranded RNA. In an additional embodiment, the construct further comprises a DNA segment coding for an oligo(dT) segment that lies between the multiple cloning site (or DNA encoding at least a portion of the gene of interest) and the DNA segment coding for the 3′-UTR complement (FIG. 2D). The oligo(dT) segment, once transcribed, hybridizes to the poly(A) segment and further lengthens the double-stranded RNA that the vector produces.

The present invention further provides a DNA construct comprising, in a 5′ to 3′ direction, a promoter, a multiple cloning site or first DNA segment encoding at least a portion of a target gene; a second DNA segment encoding a second RNA segment from a gene unrelated to the target gene; a third DNA segment encoding a third RNA segment, where the second and third DNA segments have substantial sequence identity with each other; a spacer DNA segment encoding an RNA spacer segment; a fourth DNA segment encoding a fourth RNA segment, which is complementary to the third RNA segment and forms a double-stranded RNA molecule with the third RNA segment, and a transcription terminator.

In the embodiments described above, use of the singular when referring to the target gene is not meant to limit the invention to suppression of one gene at a time. As FIG. 1C exemplifies, more than one target gene segment can be inserted into the suppression constructs to silence multiple genes simultaneously.

The suppression of gene expression, as the present invention contemplates, can be transient or stable. Transient suppression of gene expression means that the expression-targeted gene is suppressed in the organism or cell is not passed on in successive generations of the cell or organism. Suppression may, however, be passed on to one or more successive generations and still be considered transient. In contrast, stable suppression of gene expression is understood to mean that the targeted gene is suppressed in successive generations, indefinitely.

The suppression of gene expression also can be partial or complete suppression of the target gene. As used herein, suppression means that there is a reduction in the relative levels of a targeted polypeptide or protein in the host cell or organism, regardless of the mechanism of this reduction. For instance, the presence of the DNA constructs of the present invention can result in the reduction (or even complete absence) of endogenous mRNA that codes for the targeted protein, as compared to wild-type host cells or organisms. Alternatively, suppression also can mean that there is a reduction in translation of endogenous mRNA into protein, although the levels of endogenous mRNA from the target gene may not change in response to the DNA constructs. In yet another alternative, suppression can mean that the stability of the endogenous mRNA of the target gene is reduced (i.e. the MRNA is degraded more rapidly) in cells containing the DNA constructs of the present invention. As used herein, endogenous mRNA refers to the transcription product from the host cell's native, genomic DNA into an RNA molecule that is naturally occurring in a host cell or organism. The endogenous mRNA, as the present invention contemplates, can be a “mature” transcript that has been properly processed by the cell's machinery, or the transcript can be an unprocessed transcript. Additionally, the endogenous mRNA transcript need not be the complete coding sequence of a protein or polypeptide, but can be a portion thereof. As used herein, the terms protein and polypeptide are used interchangeably.

Additionally, the DNA constructs of the present invention may be sold in kit form. The kit can contain reagents to affect introduction of the DNA construct into a host of interest. In this regard, the kit can comprise sufficient amounts of the DNA construct to perform multiple cloning reactions, host cells into which the recombinant vector is introduced, one or more restriction enzymes specific for restriction enzyme sites within the DNA constructs, and one or more buffers, and various combinations thereof.

The constructs of the present invention may be used in a myriad of ways, particularly as they relate to eukaryotic biotechnology, and preferably, plant biotechnology.

The invention provides methods of producing transgenic organisms, preferably plants, with an altered genotype and, consequently, phenotype. As the present invention contemplates, the DNA constructs can be used to reduce the expression of virtually any gene of interest in a host organism. By reduction of expression is meant that there is a lower level of a targeted protein or polypeptide produced, or there is a lower activity of a targeted protein or polypeptide in the transgenic organism, compared to wild-type.

Previously identified genes of interest, or portions or promoters thereof can be isolated using methods and techniques designed for the manipulation of nucleic acid molecules, which are well known in the art. For example, methods for the isolation, purification and cloning of nucleic acid molecules, as well as methods and techniques describing the use of eukaryotic and prokaryotic host cells and nucleic acid and protein expression therein, are described by Sambrook, et al., Molecular Cloning: A Laboratory Manual, Second Edition, Cold Spring Harbor, N.Y., 1989, and Current Protocols in Molecular Biology, Frederick M. Ausubel et al. Eds., John Wiley & Sons, Inc., 1987, the disclosure of which is hereby incorporated by reference.

The DNA constructs, including at least a portion of the gene or promoter of interest, can be introduced into host cells, which as stated previously, can be individual cells, cells in culture, cells as part of a host organism, a fertilized oocyte or gametophyte or an embryonic cell. By the term “introduction,” when used in reference to a host cell is meant to refer to standard procedures known in the art for introducing recombinant vector DNA into the target host cell. Such procedures include, but are not limited to, transfection, infection, transformation, natural uptake, electroporation, biolistics and Agrobacterium.

Accordingly, the present invention also provides plants or plant cells that have been stably transformed with the constructs described herein.

Examples of genes that may be chosen for targeted suppression include, but are not limited to, those listed below. In each case, these groups of genes may include those coding for enzymes, structural proteins, or the regulatory proteins that control their expression:

Genes necessary for the normal development of flowers, cones, pollen, seeds, or fruits. (e.g., homologs of LEAFY, homologs of AGAMOUS, other MADS-box transcription factors, homologs of FT, invertase, lipid transfer protein, TA29)

Genes that inhibit flower formation (e.g. homologs of FCA and homologs of TERMINAL FLOWER1)

Genes involved in biosynthesis of secondary metabolites such as alkaloids, phenylpropanoids and isoprenoids (e.g., caffeine synthase and other methyltransferases, 4-coumarate ligase, ferulate 5-hydroxylase and other members of the P450 mono-oxygenase gene family, limonene synthase, stilbene synthase).

Genes involved in programmed cell death, including the processes of apoptosis and senescence (e.g., ACC synthase, ACC oxidase, phospholipase D, deoxyhypusine synthase, eIF5A, proteinases).

Genes involved in the differentiation and growth of xylem, such as those involved in synthesis and modification of plant cell walls (e.g., phenylpropanoid enzymes involved in lignin biosynthesis, cellulose synthase, UDP-glucose pyrophosphorylase, arabinogalactan proteins, dirigent proteins).

The following examples serve to illustrate various embodiments of the present invention and should not be construed, in any way, to limit the scope of the invention.

### EXAMPLES

The Arabidopsis ACTIN and UBIQUITIN genes whose promoters are used throughout the following examples have 5′-UTRs that contain introns. In the figures, these 5′-UTRs and introns are diagrammed as part of the promoters.

### Example 1

### Construction a Vector with a Defective Intron Segment

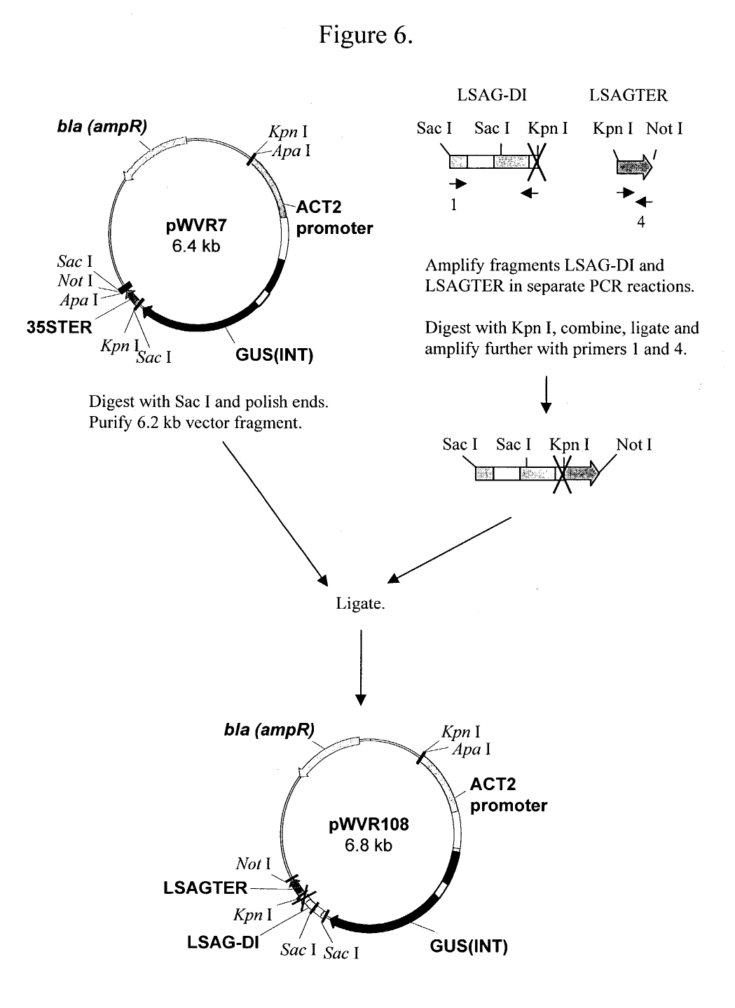

FIG. 6 depicts the method for constructing a GUS suppression vector based on a defective intron. The DNA segments of the defective intron element were derived from LSAG, a homolog of AGAMOUS isolated from sweetgum. This gene is described in U.S. patent application Ser. No. 09/433,579, which is incorporated herein by reference.

An approximately 380 bp DNA segment including exon 7, intron 7, exon 8 and the first half of intron 8 (abbreviated herein as LSAG-DI) was amplified. The 3′ primer for the amplification (5′-GAGAGAGGTACCAGTATGCATGTTT -AGGTCTTT-3′) was chosen to be upstream of the intron acceptor site, so that this acceptor site would not be present in the amplification product, and was designed to include a non-endogenous Kpn I site. The 5′ primer (EX7LSAG1, 5′-GAGAGAGAGCTCGGAATCGACTTGCACAATGATAA -3′; primer 1 in FIG. 6), located at the 5′-end of exon 7, was designed to include a non-endogenous Sac I site.

Next, an approximately 180 bp segment of terminator sequence from LSAG was amplified using the LSAG genomic DNA clone as the template. This 180 bp segment began approximately 220 bp after the translation termination codon of LSAG, and contained all poly(A) addition sites found in cDNAs examined. The 5′ primer (5′-GAGAGAGGTACCTGTTTTATGCAAGCACTACCA -3′) was designed to include a non-endogenous Kpn I site, and the 3′ primer (LSAGTER2, 5′-GAGAGAGCGGCCGCTCTAGGAGCAGCAACGATAAG-3′; primer 4 in FIG. 6) was designed to include a non-endogenous Not I site.

After digestion of each amplified fragment with Kpn I, they were combined and ligated. The ligation products were re-amplified by PCR with EX7LSAG1 and LSAGTER2 to produce a DNA segment with combining the transcribed region with the terminator. The ends of the approximately 560 bp amplification product (LSAG-DI::LSAGTER) were polished with T4 DNA polymerase.

The plasmid pWVR7 (see FIG. 6) contains a GUS(INT) sequence that comprises the full coding region from the E. coli uidA gene (described by Jefferson et al., 1987. EMBO J. 6:3901-3907) augmented with an intron from a potato tuberin gene. The GUS(INT) cassette was reported in Vancanneyt et al. (1990. Mol. Gen. Genet. 220: 245-250). pWVR7 was digested with Sac I to remove the CaMV 35S transcription terminator, and the ends of the vector DNA were subsequently made blunt using T4 DNA polymerase. The vector was purified, then ligated to the 560 bp fragment, and the resulting mixture was used to transform E. coli DH5α. In screening the colonies that were produced, two out of ten were found to have the insert.

Each clone was analyzed for proper orientation of the insert within the vector using both PCR and restriction enzyme digestion experiments. The results confirmed that LSAG exon 7 was adjacent to the GUS(INT) coding region in both plasmids and one of these was selected and designated pWVR108.

In practice, pWVR108 provides a vector with a cloning site into which many different DNA segments can be inserted. The GUS(INT) fragment can be removed using BamH I and Sac I or Nco I and Sac I, so that a promoter DNA fragment, coding DNA fragment, or population of fragments with compatible ends can be ligated into the remaining vector.

### Example 2

### Introduction of the Defective Intron Construct into Sweetgum

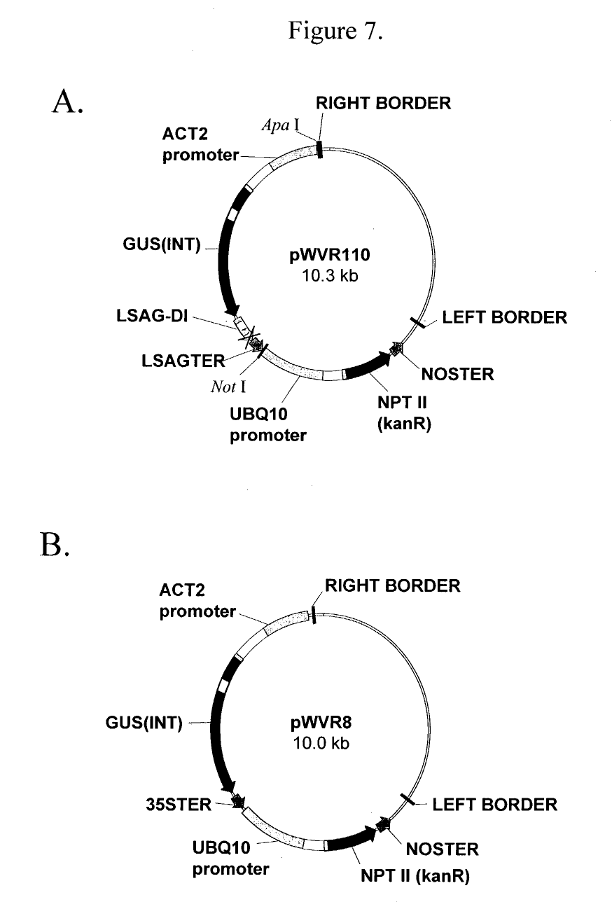

The 3.9 kb ACT2::GUS(INT)::LSAG-DI::LSAGTER cassette in pWVR108 can be excised by digestion of the vector with Apa I plus Not I in order to transfer it to a variety of other vectors, such as binary vectors for use in transformation using Agrobacterium. Plasmid pWVR110 (FIG. 7A) was produced in this manner using the compact binary vector pWVR5 (depicted at the top of FIG. 10). UBQ10 is the Arabidopsis UBIQUITIN10 gene, which provides a strong constitutive promoter for the NPTII selectable marker.

Plasmid pWVRl10 was electroporated into Agrobacterium tumefaciens strain GV2260 and a colony of kanamycin-and rifampicin-resistant bacteria was tested to verify the presence of the intact plasmid. A culture of this Agrobacterium was used to transform sweetgum.

Shoot cultures of sweetgum were maintained on Woody Plant Medium (WPM: Llyod and McCown, 1980. Proc. Int. Plant Prop. Soc. 30: 421-427) in a growth room at 25° C. with a 16-hour photoperiod. Leaf blades were harvested and maintained on SGT1 (WPM, 0.1 mg/L NAA, 2.0 mg/L BAP) medium for one to three hours until the harvest of tissue was completed or the optimal Agrobacterium density was obtained. After trimming away the leaf margins, the leaf blades were cut into 1 to 2 cm² pieces.

A 50 ml culture of Agrobacterium with plasmid pWVR 110 was grown overnight until it reached an OD₆₀₀ of 0.6-0.8. The culture was pelleted and resuspended in 50 ml of induction medium (WPM, 0.5% glucose, 2 mM NaPO₄ 50 mM MES (pH 5.0), 50 M acetosyringone). After induction of the Agrobacterium for one hour, sweetgum explants were exposed to the bacterial culture for a period of 15 minutes. The explants were then lightly blotted on sterile paper towels and placed on SGT1 medium in G7 (Magenta™) boxes. Explants were placed in at 5 per box and the boxes were maintained in the dark at 18-20° C. After a three-day co-cultivation period the explants were transferred to SGT2 medium (WPM, 0.1 mg/L NAA, 2 mg/L BAP, 500 mg/L cefotaxime) to eradicate the Agrobacterium. The explants were incubated at 25° C. in the light (250 E·m⁻²·s⁻¹) for four days.

For selection, explants were transferred to SGT4 medium (WPM, 0.1 NAA, 2 mg/L BAP, 250 mg/L cefotaxime, 75 mg/L kanamycin) at two-week intervals. Cells that grew under selection pressure were evident at approximately 2.5 months after the initiation of transformation. These calli were isolated and subcultured onto fresh SGT4 medium on a monthly basis. After adventitious shoots were initiated on this medium, the shoot clusters were transferred to SGT4.1 (WPM, 0.5 BAP, 250 mg/L cefotaxime, 75 mg/L kanamycin) for elongation. Shoots were excised after reaching 4-5 cm in length and transferred to WPM for rooting. Prior to transfer to soil, transgenic plants were propagated in vitro through axillary shoot induction on WPM medium containing 0.5 mg/L BAP.

### Example 3

### Suppression of Gene Activity in Transgenic Sweetgum

Shoots produced after co-cultivation of sweetgum leaf explants with Agrobacterium containing pWVR110 or a control plasmid were screened for the presence of the T-DNA insert using PCR to detect the NPT II selectable marker. The control plasmid was pWvR8, which differed from pWVR110 in that the 35S transcription terminator was present rather than the LSAG-DI::LSAGTER segment (see FIG. 7B). Additionally, small sections of the transgenic plants were stained for GUS activity. Twenty-three PCR-positive pWVR8 plants were tested and all showed staining. Forty-seven pWVR110 plants were tested and only seven showed staining. The presence of staining plants provided evidence that the transgene in pWVR110 was capable of being transcribed and translated to produce a functional enzyme. The fact that so few expressed the protein indicated that the gene was being silenced at a high frequency.

After culturing further on WPM medium containing 0.5 mg/L BAP for three more months to create multiple copies of each transgenic event, thirty-two of the pWVR110 lines were assayed again for GUS activity, including five of the lines that originally stained. No GUS activity above background levels was detected in any of the older plants. Silencing of transgenes as plants age is a phenomenon that has repeatedly been noted with sense suppression (e.g., Elmayan and Vaucheret, 1996. Plant J. 9: 787-797.)

### Example 4

### Construction of a Vector with an Inverted Repeat Segment

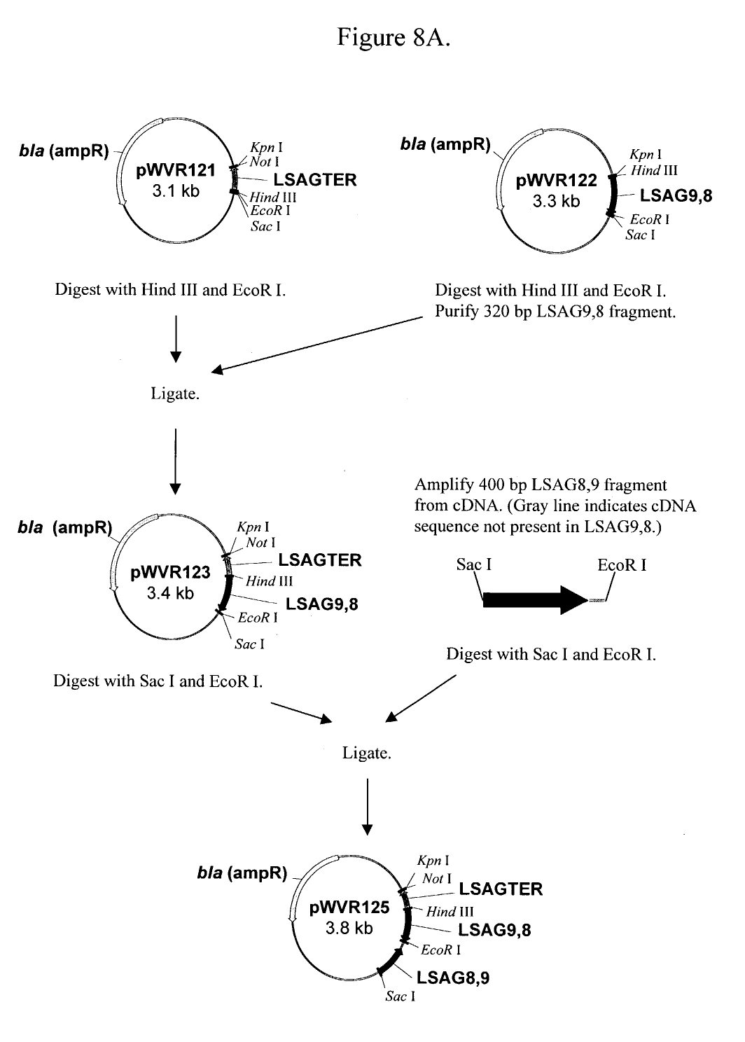

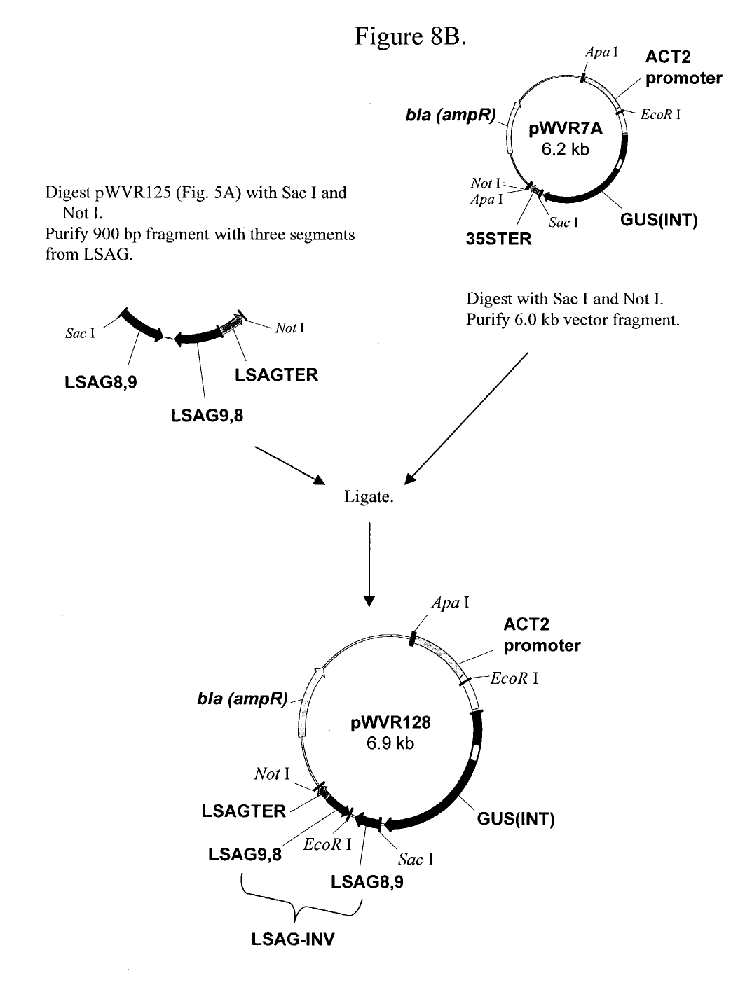

The cloning of several components of this vector is sketched out in FIG. 8A. A 140 bp segment of the LSAG terminator (LSAGTER) was amplified using PCR. This product was a subfragment of that described in Example 1, with different restriction sites introduced at the ends. The 5′ primer was designed to include a Hind III site (LSAGTER4, 5′-GAGAGAAAGCTTGCACTACCATGCTAT -AACAGATACT-3′); while the 3′ primer included both Kpn I and Not I sites (LSAGTER5, 5′-GAGAGAGGTACCGCGGCCGCATGGTTTGTAAGCCCTAAA -TTTTG-3′. The amplification product was then purified and digested with Hind III and Kpn I. After the digested product was run on a 1% agarose gel to check for proper length, it was purified and quantified. The fragment was ligated into pBluescript II SK(−) that had been digested with Hind III and Kpn I. The ligation mixture was transformed into E. coli, and colonies were screened for plasmid with insert. A plasmid was selected for further work and designated pWVR121.

Next, an approximately 320 bp portion of the LSAG eDNA including regions of exons 8 and 9 (LSAG9,8) was amplified using PCR. The 5′ primer was designed to include a non-endogenous Hind III site, and the 3′ primer was designed to include a non-endogenous EcoR I site. The amplification product was then purified and digested with EcoR I and Hind III. After the digested amplification product was run on a 1% agarose gel to verify it had the proper length, it was purified and quantified. LSAG9,8 was cloned into EcoR I- and Hind 111-digested pBluescript II to produce pWVR122.

Next, the LSAG9,8 segment and the LSAGTER segment were combined. pWVR121 was digested with Hind III and EcoR I. pWVR122 was digested with the same enzymes, and the insert was purified. Next, the prepared pWVR121 vector and 320 bp insert were combined and ligated. The ligation mixture was used to transform E. coli DH5α, and colonies were screened for the presence of the additional insertion. This procedure produced a structure where the LSAG9,8 insert was in the antisense orientation relative to the transcription terminator. This intermediate plasmid, pWVR123, was then digested with Sac I and EcoR I.

Next, DNA from a larger segment of the LSAG CDNA including regions of exons 8 and 9 was amplified using PCR. The 5′ primer included the Sac I site present in the native gene, while a non-endogenous EcoR I site was added to the 3′ primer sequence. The approximately 400 bp amplification product (referred to herein as LSAG8,9) was purified, cut with Sac I and EcoR I. After electrophoresis on a 1% agarose gel to check for proper length, the amplified DNA was extracted from the agarose and quantified.

Next, the LSAG8,9 segment was ligated into the prepared intermediate plasmid, pWVR123. As before, transformants were screened for the presence of the correct insertion. This ligation put the LSAG8,9 segment in the sense orientation relative to the transcription terminator, producing an inverted repeat of LSAG sequences (referred to herein as LSAG-INV). Because only the first 320 bp of the LSAG8,9 segment was duplicated in the LSAG9,8 segment, the non-duplicated approximately 80 bp of the sense sequence served as a spacer DNA segment (yielding the loop of the stem-loop double-stranded RNA produced after transcription and base-pairing).

The DNA segment combining LSAG-INV and LSAGTER was then used to create a complete transcription cassette, as depicted in FIG. 8B. The LSAG -INV::LSAGTER fragment was excised from plasmid pWVR125 using Sac I and Not I, and purified using gel electrophoresis. Plasmid pWVR7A, containing an ACTIN2 promoter (ACT2) and a GUS DNA segment with an intron (designated as GUS(INT)), was digested with Sac I and Not I to remove the 35STER segment and the vector fragment was purified by gel electrophoresis. The two purified fragments were ligated together and transformed into E. coli. Testing for the presence of the complete cassette was performed with restriction digests and gel electrophoresis. The structure of a selected plasmid was further verified by sequence analysis, and the plasmid was designated pWVR128.

Finally, the entire ACT2::GUS(INT)::LSAG-INV::LSAGTER cassette was excised from pWVR128 using Apa I and Not I restriction enzymes, purified, and ligated into the corresponding restriction sites of binary vector pWvR5 to produce pWVR129.

### Example 5

### Construction of Vectors with Extended Inverted Repeat Segments

For the construct in vector pWVR128, above, it is expected that the first approximately 50 bp of LSAGTER will be transcribed and a poly(A) tail will be added. Thus, the 3′OH end will be separated from the double-stranded RNA region of the transcript, and thus separated from the single-stranded sequence upstream of the stem-loop. If the presence of a 3′-OH moiety closer to the upstream single stranded RNA (which includes the target gene sequence) will increase the efficiency of suppression, this can be accomplished by placing additional sequences upstream of the inverted repeat of pWVR128. The newly inserted sequences may be linked to the remainder of the inverted repeat using standard methods of PCR amplification, restriction digestion, and ligation.

Sequence data from LSAG cDNAs showed that transcription of this gene ends immediately after . . . TATGTATTTCTT in the segment LSAGTER. Therefore, a 5′-phosphorylated PCR primer beginning with GACTCGAG (to add a Sac I site) and continuing AAGAAATACATA . . . is synthesized and used with LSAGTER4, above, to amplify the beginning of the LSAG transcription terminator segment. The approximately 70 bp amplification product is digested with Hind III, the ends are filled in using T4 DNA polymerase to create blunt ends, and gel purified. The vector pWVR128 is digested with Sac I, dephosphorylated with shrimp alkaline phosphatase, and polished with T4 DNA polymerase. After the two DNAs are mixed, ligated, and transformed into E. coli, the resulting colonies are screened for the presence of the added DNA in the correct orientation.

The PCR primer may also be designed to include bases complementary to the poly(A) tail. Thus, the 5′-phosphorylated PCR primer could begin with GACTCGAGT₍₁₀₋₂₀₎ before continuing with AAGAAATACATA . . . The PCR amplification, preparation of DNA fragments, and isolation of completed construct are done as in the preceding paragraph.

It is expected that the poly(A)-containing transcripts produced from either of these constructs after transformation into a host cell will have their poly(A)-tracts trimmed from the 3′-ends until eventually the 3′-OH is flush with the single-stranded RNA of the transcript. Then, if the suppression mechanism involves an RNA-dependent RNA polymerase that requires priming from the double-stranded region, there should be a greater frequency or efficiency of suppression.

### Example 6

### Construction of Vector with Three Linked Copies of Repeated Segment (Two Copies in an Inverted Repeat)

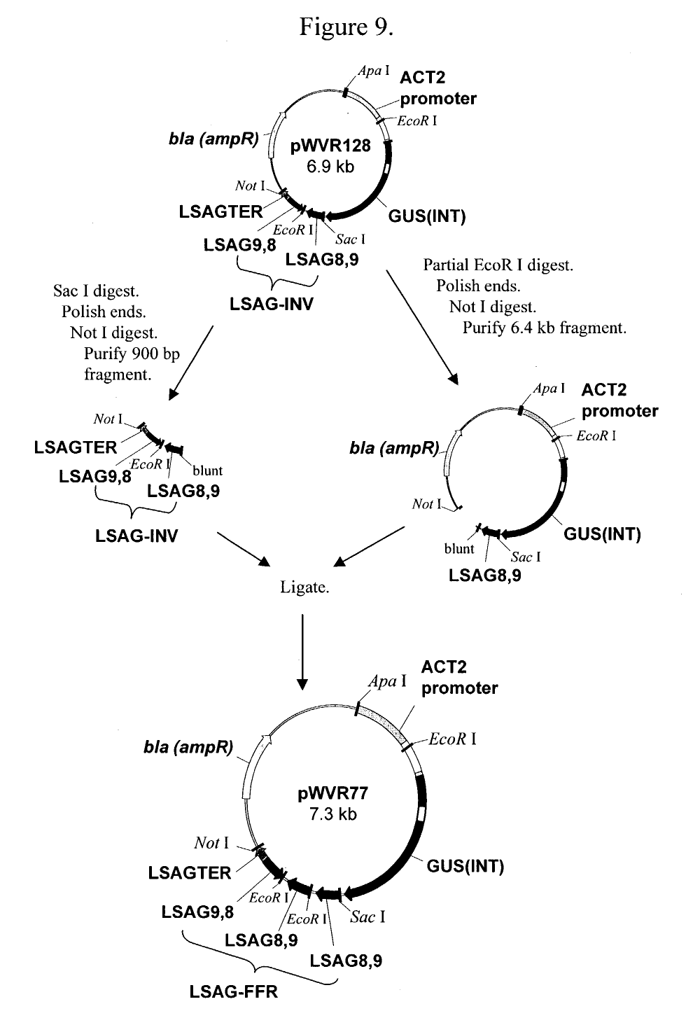

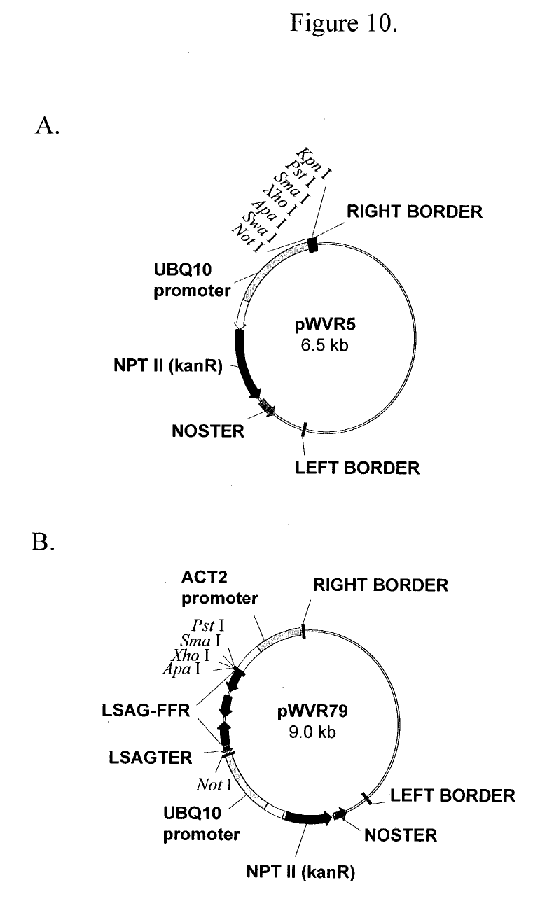

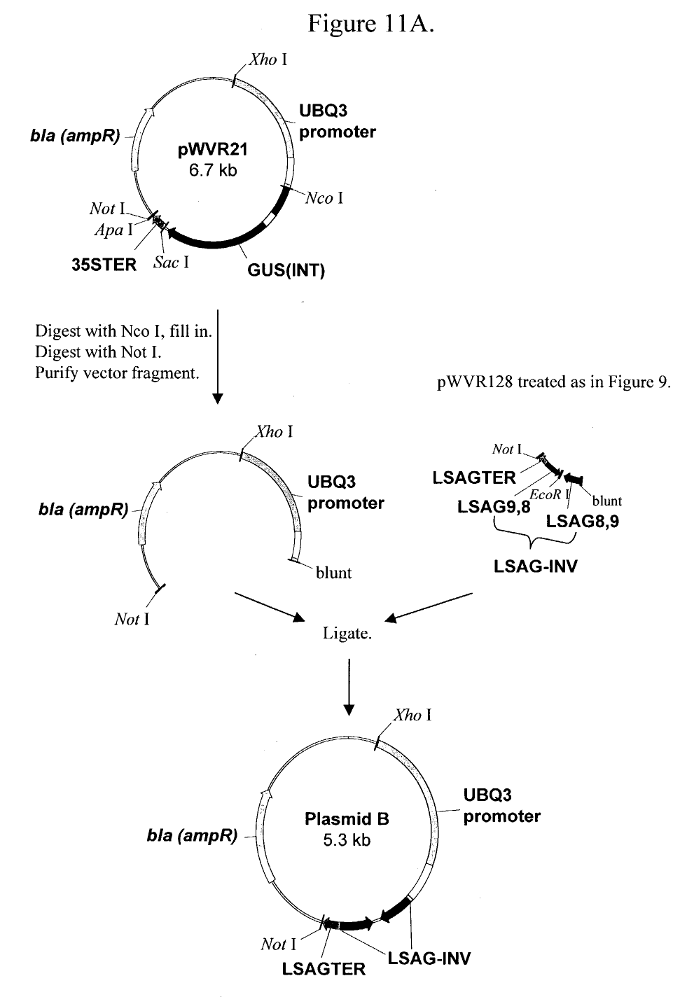

This vector links a single-stranded region of the LSAG gene to the segment from the target gene, and additionally comprises a stem-loop of the same LSAG sequence (FIG. 9).

A first sample of plasmid pWVR128 was partially digested with EcoR I, the ends were filled in with T4 DNA polymerase, and the linearized approximately 6.9 kb product was isolated using gel electrophoresis. The isolated DNA was then digested with Not I, and the products were again resolved using gel electrophoresis. The 6.4 kb vector fragment resulting from the removal of the approx. 500 bp LSAG9,8::LSAGTER segment was isolated.

A second sample of plasmid pWVR128 was digested with Sac I, and the ends were polished with T4 DNA polymerase. After removal of the polymerase by phenol extraction, ethanol precipitation of the plasmid, and resuspension, the DNA was then digested with Not I and the approximately 900 bp LSAG-INV:: LSAGTER segment was purified using gel electrophoresis.

The DNA fragments resulting from the first and second samples were mixed in a 1::3 mass ratio, ligated and transformed into E. coli strain XL10. The resulting colonies are screened for the presence of three copies of the LSAG sequence. Sac I-, Not I-double digestion yielded bands of 6.0 and 1.3 kb, and EcoR I digestion produced bands of 4.1, 2.8 and 0.4 kb for the correct construct designated pWVR77. The LSAG8,9::LSAG-INV combination is referred to herein as LSAG-FFR. After transcription, the RNA corresponding to the LSAG-FFR region will have a single-stranded and a double-stranded portion.

### Example 7

### Use of LSAG-FFR Vector to Perform Functional Screening of a cDNA Library

Functional testing of genes by “knock-outs” that disrupt gene activity is a common practice in molecular genetics research on a variety of plant, fungal, and animal species. In plants, random knockouts have been accomplished by insertion of transposable elements or T-DNA. If a researcher's interest lies in a specific developmental process, the random nature of these methods makes them very inefficient and thus poorly applicable to species that are larger or mature more slowly than Arabidopsis. Viral-based suppression of genes has provided a partial solution to this, because cDNAs from a selected developmental state can be inserted into the viral vector for testing. But in systems where the plant needs to grow outdoors due to size constraints or the need for environmental exposure, government regulations can be a major obstacle to the use of viral suppression vectors. A binary plasmid bearing the LSAG-FFR cassette can be used to solve this problem in species that are susceptible to transformation by Agrobacterium.

The overall strategy used in this example is a) production of a binary vector suitable for accepting the cDNA inserts, b) synthesis of the cDNA, c) insertion of cDNAs into the vector to make a library, d) transformation of the recombinant vector library into Agrobacterium, e) generation of transformed plants, f) evaluation of plants and selection of phenotypes of interest, and g) identification of the CDNA associated with the phenotype for further testing.

The primary modifications necessary to convert pWVR5 (FIG. 10A) into a suitable binary vector (FIG. 10B) are the addition of the promoter, the addition of the LSAG-FFR::LSAGTER segment, and (optionally) the addition of restriction sites compatible with the cDNA segments to be inserted.

First, pWVR5 is digested with Kpn I. The linearized vector is treated with T4 DNA polymerase to make the ends blunt, and treated with phosphatase. A sample of pWVR77 (described in Example 6 and in FIG. 9) is digested with Apa I and Nco I, and the digestion products treated with T4 DNA polymerase to make the ends blunt. The 1.2 kb ACT2 promoter fragment is purified by gel electrophoresis. The vector and promoter DNA preps are mixed, ligated and transformed into E. coli. The resulting colonies are screened using PCR for the presence of a plasmid with the promoter in the correct orientation (former Apa I site next to the Pac I site and former Nco I site next to the Pst I site). Primers ACT2SEQ1 (5′-GAACACTGTTTAAGTTAGATGAAG-3′) in the promoter and UBQPR2 (5′-CAATAGCTAATAATAAAATGACAC-3′) in the T-DNA selectable marker produce a 1.0 kb product when this DNA arrangement is present. The new plasmid A is digested with Swa I, then digested with Not I. A second sample of plasmid pWVR77 is digested with Sac I, treated with T4 DNA polymerase to make the ends blunt, and the polymerase removed by phenol extraction. The plasmid is then digested with Not I and the 1.3 kb LSAG-FFR::LSAGTER fragment is isolated. The prepared fragments are ligated together and the mixture transformed into E. coli. Colonies are selected and screened for the presence of the correct structure. The selected new binary plasmid is designated pWVR79. It has a multiple cloning region with unique restriction sites (Pst I, Sma I, Xho I, Apa I) between the promoter and LSAG-FFR segments. Other restriction sites can be substituted for these by methods known to those skilled in the art.

A variety of methods for synthesis of a cDNA library from selected tissue or developmental state are known to those skilled in the art (e.g., Sambrook et al., 1989). For functional screening, it is preferred that the cDNA library be subtracted, or depleted of sequences that are expressed in tissues other than the one of interest (e.g., Duguid et al., 1988. PNAS 85: 5738-5742; Hedrickson et al., 1994. Nature 308: 149-153.). For example, a library from reproductive tissues could be subtracted against cDNA from vegetative tissues. A library from developing xylem could be subtracted against eDNA from cambium and phloem tissues, and a library from hormone-treated roots could be subtracted against cDNA from roots that were not treated. It is also preferred that the library be normalized or equalized (treated so that sequences from highly expressed genes are reduced to a smaller portion of the total cDNA population; Soares and Efstratiadis, 1998. U.S. Pat. No. 5,830,662.). BD-Clontech sells the PCR-Select™ cDNA kit, which yields cDNA libraries with both of these properties. This kit produces cDNA fragments with blunt ends, which must be ligated into a vector with blunt, phosphatase-treated ends, thus not allowing the orientation of the inserted cDNAs to be predetermined. The Stratagene Lambda-ZAP™ cDNA kit produces segments with an EcoR I site at the 5′-end and an Xho I site at the 3′-end. This and other kits can be supplemented with custom-designed oligonucleotides to create cDNAs with, for example, Pst I and Xho I ends. For the purposes of this example, it is assumed that blunt-ended cDNAs will be inserted into the Sma I site of the vector. However, this is not meant to limit in any way the scope of the invention to use of this particular cloning method.

The CDNA mixture produced by the Clontech kit is digested with Rsa I to create blunt ends, then ligated into pWVR79 that has been prepared by Sma I digestion and phosphatase treatment. The resulting mixture is transformed into a selected Agrobacterium strain such as GV2260 using electroporation. After plating on selective medium, the individual colonies represent random CDNA fragments inserted into the suppression vector. Some clones may have multiple insertions, but this is acceptable. Agrobacterium colonies are selected and pooled in groups of 100. These pools are used to prepare bacterial suspensions to co-cultivate with plant material for transformation. If 1000 cDNA clones are being used, ten separate pools are created and ten plant transformation procedures are done.

Preferably, the plant material used is from a species that is the same as or is closely related to the species that was used to make the CDNA. This assures sufficient sequence similarity for the suppression mechanism to function. After co-cultivation with an Agrobacterium pool, the plant material is cultured on medium that selects against further bacterial growth and selects for the presence of transformed plant cells (e.g., containing cefotaxime to kill bacteria and kanamycin to select for the T-DNA marker, as described in Example 2, above). The specific media and transfer protocols will depend upon the species being transformed. If hybrid poplar is being transformed, a suitable protocol can be found in Leple et al. (1992. Plant Cell Rep. 11: 137-141.). For each pool of cDNA clones, 100 to 1000 independently transformed shoots are generated. Phenotype changes due to the suppression of plant genes may be visible as soon as transformed shoots begin to develop. The particular shoots selected for further study are grown in vitro and in soil (perhaps outdoors) to collect phenotypic data.

The identity of the cDNA(s) present in the selected plants is determined by using PCR to isolate the insert sequences from the plants' genomic DNA. Primers flanking the insert, such as ACT2SEQ1 (in the promoter) and LSAGEX8R (5′-TCAGGCCCATTAAACTAATTGAAG-3′, in the repeat unit) can be used in the amplification. The amplified band(s) can be cloned for further sequence analysis, for example by digesting with Pst I and Xho I and ligation into pBluescript SK that has been cut with the same enzymes. Some plants will have multiple inserts that need to be further analyzed to determine which is responsible for the observed phenotype. Such analyses include new transformations with a suppression construct bearing the isolated sequence as the target sequence, rather than a mixture.

### Example 8

### Synthesis of a Combination of Constructs with Three Copies of Repeated Segment (Inverted Repeat in Separate Cassette)

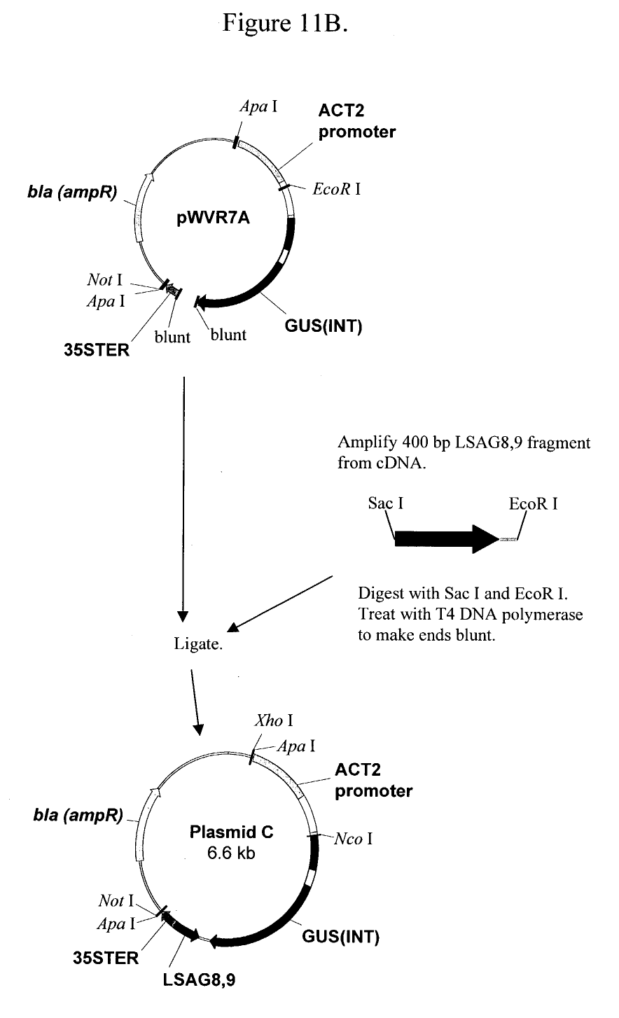

The production of an inverted repeat cassette is diagrammed in FIG. 11A. A sample of plasmid pWVR21 is digested with Nco I and the DNA is treated with T4 DNA polymerase to make the ends blunt. Then the DNA is cut with Not I and the vector fragment is purified. then the DNA is ligated and transformed into E. coli. The resulting colonies are screened for the correct structure. UBQ3 is the Arabidopsis UBIQUITIN3 promoter (sequence in GenBank Accession number L05363), which provides a strong constitutive promoter for the inverted repeat. Plasmid B will not be digestible with Nco I, Sac I, Apa I, nor BamH I. It will be linearized to a 5.3 kb fragment by digestion with Xho I, and will produce 2.7 and 2.6 kb fragments when digested with Xho I plus Not I.

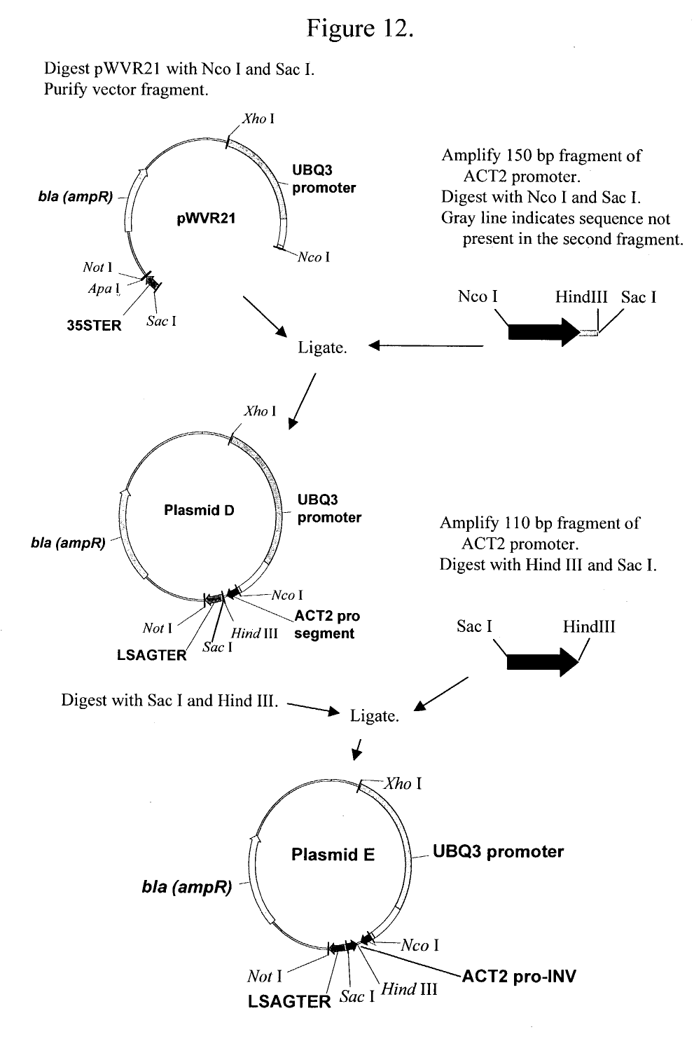

Construction of the target linked to a single segment of the repeat unit is diagrammed in FIG. 12B. Plasmid pWVR7A is digested with Sac I, the ends are made blunt by treatment with T4 DNA polymerase, and the ends are treated with phosphatase. Fragment LSAG8,9 is amplified as described in Example 4. To make sure that ends of the fragment are phosphorylated, the fragment is digested with Sac I and EcoR I, then treated with T4DNA polymerase. The LSAG8,9 fragment is then ligated into the prepared plasmid and the mixture transformed into E. coli. Colonies are then screened for the presence of the additional segment, yielding a construct with GUS as the target segment, plasmid C. Either orientation of the LSAG8,9 segment is acceptable.

The two high-copy plasmids may be used in biolistic transformations, but two more steps are required for Agrobacterium-based transformation. 1) Binary vector pWVR5 (FIG. 10A) is digested with Sma I and the ends treated with phosphatase. Plasmid C is cleaved with Apa I, the ends are polished by treating with T4 DNA polymerase and the ACT2::GUS(INT)::LSAG8,9:: 35STER cassette is purified. The cassette is then ligated into the prepared binary plasmid and transformed into E. coli. 2a) The intermediate plasmid resulting from this is cleaved with Xho I and Not I. Next, plasmid B is cleaved with the same enzymes and the UBQ3::LSAG-INV::LSAGTER cassette is purified. This cassette is then ligated into the prepared intermediate plasmid and transformed into E. coli. After various tests such as PCR, restriction digestion and sequencing, a preparation of the plasmid with the correct structure (see FIG. 4C) is transformed into Agrobacterium, which is subsequently used in plant transformation. The plants resulting from this transformation can be crossed to GUS-positive plants containing the T-DNA of pWVR8 (see FIG. 7B) to demonstrate silencing of GUS in the offspring.

Alternatively, 2b) Plasmid B is cleaved with Xho I and Not I, and the UBQ3::LSAG-INV::LSAGTER cassette is purified. This fragment is ligated into a sample of the pWVR5 that was digested using the same enzymes and the mixture is transformed into E. coli. Upon verification of the structures, the plasmids from step 1 and step 2b are individually transformed into Agrobacterium. The two Agrobacterium cultures bearing the separate plasmids are mixed for use in a co-transformation of plant tissues. Because the two plasmids have the same selectable marker, it will be necessary to screen regenerated transformed shoots using PCR to verify that both components of this combination are present.

Two considerations may cause this embodiment of the invention to be preferred over the triple repeat construct described in Example 6. First, these plasmids may be more stable in bacteria because they have a less complex repeat structure. Second, the transcripts produced by the target::LSAG8,9 cassette of this embodiment may be better able to interact with the suppression machinery because they are less likely to be degraded by other mechanisms. Transcripts produced by the target::LSAG-FFR cassette will be cleaved rapidly at the double-stranded RNA region, potentially leaving the remainder of the transcript to be degraded by housekeeping ribonucleases and thus possibly resulting in a lower effective concentration of the target::LSAG fusion in the cell.

### Example 9

### Construction of Vector with Target Promoter Segments as an Inverted Repeat

The selected promoter fragment is amplified from genomic DNA or from a plasmid using two pairs of primers. For silencing the Arabidopsis ACT2 promoter (Genbank Accession Number U41998) as diagrammed in FIG. 12, the first 5′ primer (5′-GAGAGACCATGGCTGTCTGACAGCCAGGTCAC-3′) adds a non-endogenous Nco I site to the 5′-end of the sequence and the first 3′ primer (5′-GAGAGAGAGCTCAAAAGCTTCAAAGCGGAGAGGAAAATATATG-3′) adds non-endogenous Hind III and Sac I sites. After amplification, the fragment is digested with Nco I and Sac I, then is purified by gel electrophoresis. Plasmid pWVR7A is cleaved with the same enzymes and the 4.2 kb vector fragment is purified. The treated vector and PCR product are ligated to each other and transformed into E. coli. The resulting colonies are screened for the presence of intermediate plasmid D, which has the GUS coding sequence removed and the approximately 150 bp promoter fragment present.

The second amplification of a promoter fragment uses a second 5′ primer (5′-GAGAGAGAGCTCTGTCTGACAGCCAGGTCAC-3′) that adds a non-endogenous Sac I site to the PCR product. The second 3′ primer in the amplification (5′-GAGAGAAGCTTGGCGGGTTTATCTCTTACAACTTTA-TTTTCG-3′) adds a non-endogenous Hind III site to the 3′-end of the fragment. After amplification, the approximately 110 bp fragment is digested with Sac I and Hind III and purified. Plasmid D is digested with the same enzymes, then the prepared plasmid and fragment are ligated to each other, and the mixture is used to transform E. coli. The resulting colonies are screened for the presence of the correct plasmid, based on restriction digestion and PCR. The correct structure will have the second insert in a reversed orientation relative to the first insert (plasmid E in FIG. 12).

For other promoters, the spacer segment between the two units of the inverted repeat may be formed in a variety of ways. The fragments amplified by the two sets of primers preferably differ from each other at their 3′-ends. One fragment may be longer than the other, so that non-repeated promoter sequence serves as spacer. Alternatively, if the spacer need only be 10 to approximately 50 bp, the 3′-primers can comprise synthetic sequences that lie between the 3′-restriction sites and the promoter sequence. Longer spacers, if required, can be inserted in a separate cloning step after formation of plasmid D using the same basic methods. If the promoter segments contain endogenous restriction sites that interfere with the procedures outlines here, other restriction enzymes can readily be substituted at the ends of the amplified fragments.

The activity of the promoter may be modulated rather than completely silenced if the inverted repeat comprises a small fragment of the promoter (e.g., 30-50 bp). Regulatory motifs identified by sequence analysis or other means can be targeted with this method. Transcription factor binding sites, enhancer sequences and silencer sequences located at a distance from the transcription start site are possible targets. Also, the promoter may be scanned for effective target sequences by creating a set of suppression constructs with overlapping target segments to be tested individually.

### Example 10

### Introduction of the FFR Construct into Tobacco

Binary vectors were constructed comprising ACT2::GUS(INT)::LSAG-FFR:: LSAGTER, ACT2::GUS(INT)::LSAG-INV::LSAGTER and ACT2::GUS(INT)::LSAGTER. The 4.5 kb cassette from pWVR77 (FIG. 9) was excised by digestion with Apa I plus Not I, gel purified, and ligated into a binary vector for use in transformation using Agrobacterium. The resulting plasmid was named pAGF1O. Likewise, the 4.1 kb cassette in pWVR128 (FIG. 8B) was excised using the same enzymes and put into the binary vector to produce pAGF13.

The control reporter plasmid, pAGF14, was constructed in two steps. First, the plasmid pWVR128 (FIG. 8B) was digested with Sac I, then partially digested with Hind III, then the ends were made blunt using T4 DNA polymerase plus dNTPs. The 6.1 kb fragment resulting from elimination of the LSAG-INV inverted repeat was gel purified and re-circularized with T4 DNA ligase to produce pWVR131. pAGF14 was produced by excising the 3.5 kb GUS cassette of pWVR131 using Pst I and Not I, gel purifying it, and ligating it into the binary vector.

Plasmids pAGF10, pAGF13, and pAGF14 were individually electroporated into Agrobacterium tumefaciens strain GV2260 and, for each, a colony of kanamycin- and rifampicin-resistant bacteria was tested to verify the presence of the intact plasmid. Cultures of these Agrobacterium were used to transform tobacco (Horsch et al. 1985. Science. 227:1229-1231.)

### Example 11

### Preliminary Evidence for Suppression of Gene Activity in Transgenic Tobacco

Shoots produced after co-cultivation of tobacco leaf explants with Agrobacterium containing pAGF10, pAFG13 or pAGF14 were screened for the presence of the T-DNA insert using PCR to detect the selectable marker. Twenty-four independent plants were produced for each construct. Approximately one month after transfer of transformed plants into soil, and again as the plants were flowering, samples were taken to determine the activity of the GUS reporter gene. Slices of petioles 2-3 mm long were sampled from leaves near the middle of the plants at the earlier time, and within the inflorescences at the later time. Samples were placed in 200 μL of buffered X-Gluc (Jefferson et al., 1987. EMBO J. 6:3901-3907.), incubated under vacuum for one hour, then incubated at 37 C for approximately 20 hrs. Chlorophyll pigment was removed by soaking petiole slices in methanol twice, for 12 hrs each time.

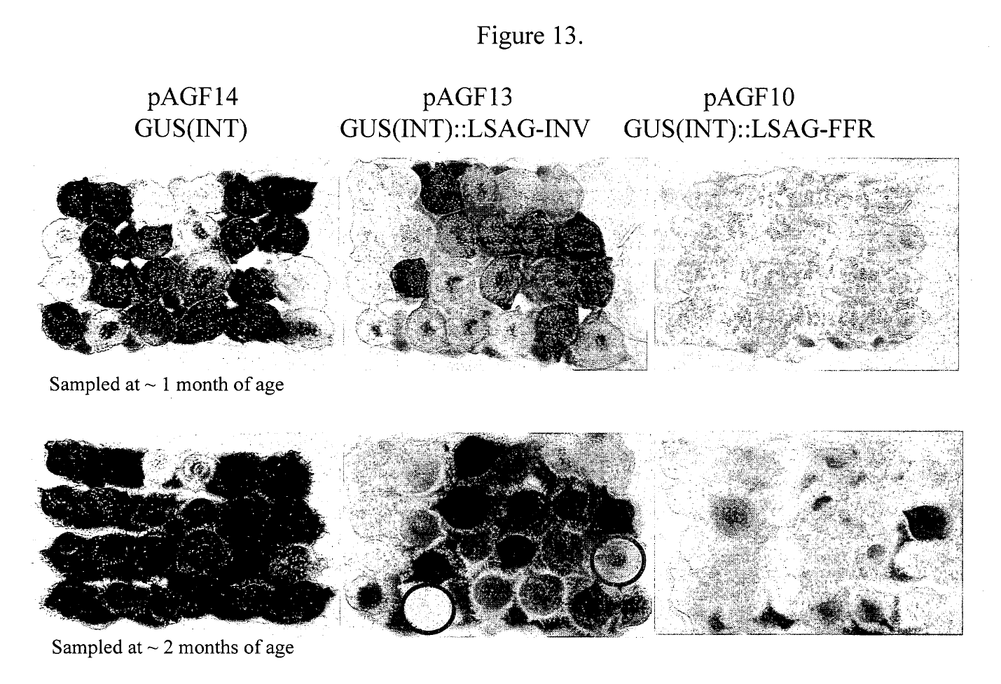

FIG. 13 shows the results of the staining experiment. For each plasmid, the samples are arrayed so that the same plants are in the same position for both time points. pAGF14, the control, showed moderate to strong expression in about half of the younger plants and in almost all of the older plants. pAGF13, the construct with the simple inverted repeat of the LSAG sequence, showed a reduced level of expression relative to the control at both sampling times. Two of the plants bearing pAGF13 (circled samples in FIG. 13) appear to have activated suppression as the plants aged. Plants bearing pAGF10, with the FFR repeat, had a much lower frequency of moderate or high expression than those bearing either of the other two plasmids. The data supports the idea that the FFR repeat leads to instability of the linked sequences. The ability of FFR repeat to silence other genes containing the target GUS sequence can be confirmed by crossing pAGF10 plants with plants expressing GUS, such as the pAGF14 controls. Silencing of GUS expression should be seen in progeny inheriting both of the transgenes. The pAGF10 plants and their progeny also could be analyzed for the presence of the 21-24 nt siRNAs that are considered diagnostic for RNA interference (Hamilton and Baulcombe. 1999. Science. 286: 950-952.)

What is claimed is:

1. A DNA construct comprising a promoter operably linked to
a first DNA segment that corresponds to at least a portion of a target gene,
a second DNA segment that corresponds to at least a portion of a gene different from the target gene,
a third DNA segment that has substantial sequence identity to the second DNA segment,
a spacer, and
a fourth DNA segment that is the reverse complement of at least a portion of the third DNA segment,
wherein the first, second, third and fourth DNA segments are arranged in a 5′ to 3′ direction, respectively, in the DNA construct.

2. An expression system comprising
A) a first DNA cassette comprising a first DNA segment that corresponds to at least a portion of a target gene and a second DNA segment that corresponds to at least a portion of a gene different from the target gene, wherein the first and second DNA segments are arranged in a 5′ to 3′ direction, respectively, in the first DNA cassette,
B) a second DNA cassette comprising a third DNA segment that has substantial sequence identity to the second DNA segment of the first DNA cassette, a spacer and a fourth DNA segment that is the reverse complement of at least a portion of the third DNA segment, wherein the third and fourth DNA segments are arranged in a 5′ to 3′ direction, respectively, in the first DNA cassette.

3. The expression system of claim 2, wherein the first and second DNA cassettes are in the same construct.

4. The expression system of claim 2, wherein the first and second DNA cassettes are in separate constructs.

5. The DNA construct of claim 1, where said first DNA segment comprises a sense sequence.

6. The DNA construct of claim 1, where said target gene is a promoter of gene of interest.

7. A DNA construct comprising a promoter operably linked to a first DNA segment that corresponds to at least a portion of a promoter of a target gene, a spacer, and a second DNA segment that is the reverse complement of at least a portion of the first DNA segment,
wherein the first and second DNA segments are arranged in a 5′ to 3′ direction, respectively, in the DNA construct.

8. A method of suppressing the expression of a target gene in a cell comprising introducing into the cell the DNA construct of claim 1.

9. A method of suppressing the expression of a target gene in a cell comprising introducing into the cell the expression system of claim 2.

10. A method of suppressing the expression of a target gene in a cell comprising introducing into the cell the DNA construct of claim 7.

11. The method of claim 8, wherein the cell is a plant cell.

12. A transformed plant cell comprising the DNA construct of claim 1.

13. A transgenic plant comprising the DNA construct of claim 1.

14. A kit for suppressing gene expression comprising the DNA construct of claim 1 and reagents to affect introduction of the DNA construct into a host of interest.


SEQUENCE LISTING (14 sequences)

SEQ ID NO: 1 - DNA, 33 bp, Artificial Sequence
  Description of Artificial Sequence Primer
gagagaggtaccagtatgcatgtttaggtcttt

SEQ ID NO: 2 - DNA, 35 bp, Artificial Sequence
  Description of Artificial Sequence Primer
gagagagagctcggaatcgacttgcacaatgataa

SEQ ID NO: 3 - DNA, 33 bp, Artificial Sequence
  Description of Artificial Sequence Primer
gagagaggtacctgttttatgcaagcactacca

SEQ ID NO: 4 - DNA, 35 bp, Artificial Sequence
  Description of Artificial Sequence Primer
gagagagcggccgctctaggagcagcaacgataag

SEQ ID NO: 5 - DNA, 12 bp, Artificial Sequence
  Description of Artificial Sequence Synthetic oligonucleotide
tatgtatttctt

SEQ ID NO: 6 - DNA, 12 bp, Artificial Sequence
  Description of Artificial Sequence Synthetic oligonucleotide
aagaaatacata

SEQ ID NO: 7 - DNA, 28 bp, Artificial Sequence
  Description of Artificial Sequence Primer
gactcgagtttttttttttttttttttt

SEQ ID NO: 8 - DNA, 24 bp, Artificial Sequence
  Description of Artificial Sequence Primer
gaacactgtttaagttagatgaag

SEQ ID NO: 9 - DNA, 24 bp, Artificial Sequence
  Description of Artificial Sequence Primer
caatagctaataataaaatgacac

SEQ ID NO: 10 - DNA, 24 bp, Artificial Sequence
  Description of Artificial Sequence Primer
tcaggcccattaaactaattgaag

SEQ ID NO: 11 - DNA, 32 bp, Artificial Sequence
  Description of Artificial Sequence Primer
gagagaccatggctgtctgacagccaggtcac

SEQ ID NO: 12 - DNA, 43 bp, Artificial Sequence
  Description of Artificial Sequence Primer
gagagagagctcaaaagcttcaaagcggagaggaaaatatatg

SEQ ID NO: 13 - DNA, 31 bp, Artificial Sequence
  Description of Artificial Sequence Primer
gagagagagctctgtctgacagccaggtcac

SEQ ID NO: 14 - DNA, 42 bp, Artificial Sequence
  Description of Artificial Sequence Primer
gagagaagcttggcgggtttatctcttacaactttattttcg